(19) United States
(12) Patent Application Publication
(10) Pub. No.: US 2017/0219501 A1
(43) Pub. Date: Aug. 3, 2017

(54) METHODS AND SYSTEMS FOR TIME-OF-FLIGHT X-RAY TOMOGRAPHY

(71) Applicant: THE RESEARCH FOUNDATION FOR THE STATE UNIVERSITY OF NEW YORK, Albany, NY (US)

(72) Inventors: Michael Yakimov, Ballston Lake, NY (US); Serge Oktyabrsky, Rexford, NY (US)

(73) Assignee: THE RESEARCH FOUNDATION FOR THE STATE UNIVERSITY OF NEW YORK, Albany, NY (US)

(21) Appl. No.: 15/416,948

(22) Filed: Jan. 26, 2017

Related U.S. Application Data
(60) Provisional application No. 62/288,303, filed on Jan. 28, 2016.

(51) Int. Cl. G01N 23/203; A61B 6/00; A61B 6/03

(52) U.S. Cl. CPC G01N 23/203; A61B 6/032; A61B 6/4208; G01N 2223/419; G01N 2223/506

(57) ABSTRACT

A system for Time-of-Flight tomography includes an x-ray source capable of producing pulsed x-rays with a pulse duration of about 100 ps or faster, a single-photon detector configured to detect individual photons backscattered from an object when present and illuminated by the x-ray source, the single-photon detector producing a two-dimensional image, and a processor for determining a Time-of-Flight of an individual photon from the x-ray source and backscattered by the object to the single-photon detector. Operating the system pulsing at 100 picosecond or faster, pulsing the x-ray source at least once to illuminate at least part of an object, detecting via a detector one or more individual backscattered photons from the object, and determining a length of time for an individual backscattered photon to travel from the x-ray source to the photon detector.

### CROSS-REFERENCE TO RELATED APPLICATIONS

This application claims priority under 35 U.S.C. §119 to U.S. Provisional Application No. 62/288,303, filed Jan. 28, 2016, which is herein incorporated by reference in its entirety.

### BACKGROUND OF THE INVENTION

Technical Field

The present invention generally relates to x-ray imaging. More particularly, the present invention relates to x-ray imaging by detecting individual backscattered photons and determining a Time-of-Flight (elapsed time from emission to detection) thereof.

Background Information

Currently, three-dimensional imaging can be done in a number of ways. For example, one type of system uses direct reflection of a pulsed laser (pulse width about 100 picoseconds) with photon counting via a silicon avalanche photodiode matrix and a start-stop time correlator. Another example of conventional three-dimensional imaging is computer tomography (CT) scanning, where an x-ray source and detectors are situated on opposite sides of an object being imaged, and rotated about the object until enough images are taken, for example, hundreds of images. In CT scanning, the imaging provides a two-dimensional image and the depth information is computed from the multiple images (projections). However, each system has its drawbacks, for example, the number of images (and cumulative dose) needed for CT scanning of people presents significant health risk.

Thus, a need continues to exist for new three-dimensional imaging methods and systems, especially applicable to medical imaging.

### SUMMARY OF THE INVENTION

The shortcomings of the prior art are overcome and additional advantages are provided through the provision, in one aspect, of a method of three-dimensional imaging. The method includes providing an x-ray source configured to produce pulsed x-rays, each pulse having a time duration of about 100 ps or faster, pulsing the x-ray source at least once to illuminate at least part of an object, detecting via a detector one or more individual backscattered photons from the object, and determining a length of time for an individual backscattered photon to travel from the x-ray source to the photon detector.

In accordance with another aspect, a system for Time-of-Flight tomography is provided. The system includes an x-ray source capable of producing pulsed x-rays with a pulse duration of about 100 ps or faster, a single-photon detector configured to detect individual photons backscattered from an object when present and illuminated by the x-ray source, the single-photon detector producing a two-dimensional image, and a processor for determining a Time-of-Flight of an individual photon from the x-ray source and backscattered by the object to the single-photon detector.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1 depicts a simplified example of a Time-of-Flight (TOF) x-ray tomography setup to illustrate the operating principles, in accordance with one or more aspects of the present invention.

FIG. 2 depicts one example of a system for Time-of-Flight (TOF) x-ray tomography with relatively small doses and relatively slow imaging, in accordance with one or more aspects of the present invention.

FIG. 3 depicts one example of a single-photon detector with pixel array, in accordance with one or more aspects of the present invention.

FIG. 4 depicts one example of an x-ray pulse showing start/stop times for determining a Time-of-Flight of a given photon, in accordance with one or more aspects of the present invention.

FIG. 5 depicts one example of a graph of distribution of start/stop times (TOFs), in accordance with one or more aspects of the present invention.

FIG. 6 depicts one example of a distribution of TOFs of backscattered x-ray photons, in accordance with one or more aspects of the present invention.

FIG. 7 depicts another example of a TOF imaging system with a high dose and relatively fast imaging, in accordance with one or more aspects of the present invention.

FIG. 8 depicts a third example of a TOF imaging system with a relatively moderate dose and relatively moderate imaging speed, in accordance with one or more aspects of the present invention.

FIG. 9 depicts one example of a scattering point of an object being imaged, with a collimator and the TOF detector of the example of FIG. 7, useful for an object comparable in size to the detector, and a fan-like collimator useful for objects significantly larger than the size of the detector.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the invention in detail. It should be understood, however, that the detailed description and the specific examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

Approximating language, as used herein throughout the specification and claims, may be applied to modify any quantitative representation that could permissibly vary without resulting in a change in the basic function to which it is related. Accordingly, a value modified by a term or terms, such as “about,” is not limited to the precise value specified. In some instances, the approximating language may correspond to the precision of an instrument for measuring the value.

The terminology used herein is for the purpose of describing particular examples only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include (and any form of include, such as “includes” and “including”), and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more steps or elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

As used herein, the term “connected,” when used to refer to two physical elements, means a direct connection between the two physical elements. The term “coupled,” however, can mean a direct connection or a connection through one or more intermediary elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable or suitable. For example, in some circumstances, an event or capacity can be expected, while in other circumstances the event or capacity cannot occur—this distinction is captured by the terms “may” and “may be.”

As used herein, unless otherwise specified, the term “about” used with a value, such as measurement, size, etc., means a possible variation of plus or minus twenty percent of the value.

As used herein, the term “three-dimensional pixel” refers to a data set (hereinafter, “three-dimensional image”) having an array of elements, where information recorded by each element includes information regarding the magnitudes of x-ray backscattering at different positions along a given line within an analyzed volume of an object. As used herein, the term “N×M three-dimensional image” refers to N×M array of three-dimensional pixels containing N×M×D individual values, each three-dimensional pixel having D individual values describing three-dimensional distribution of x-ray scattering magnitude within the analyzed volume, “D” being dependent on the temporal resolution of the single-photon detector and pulse duration of the x-ray source.

Reference is made below to the drawings, which are not drawn to scale for ease of understanding, wherein the same reference numbers are used throughout different figures to designate the same or similar components.

In conventional Computer Tomography (CT) Scanning, the x-ray source and detectors are situated opposite each other with the object being scanned situated in between the source and detectors, which move around the object. Two-dimensional information about the object is obtained by the detectors, while depth information is obtained from multiple (typically, hundreds) of images.

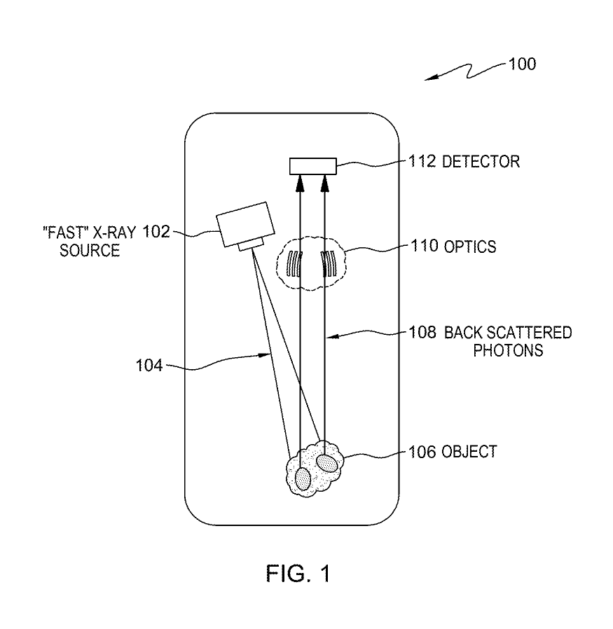

In contrast, a Time-of-Flight (TOF) system 100 can use as few as a single image. As shown in FIG. 1, a relatively “fast” x-ray source 102 emits an x-ray pulse 104 that impinges on an object 106 and results in backscattered photons 108 that, together with the use of optional optics 110, impinge on a single-photon detector 112. Compton scattering (versus absorption in a conventional CT) accounts for the majority of backscattered photons, and is necessary to obtain enough information to image the object of interest. In one example, two-dimensional information is obtained by the detector, while depth information is obtained by measuring the time for a scattered photon to travel from the source to the detector. With the TOF information, a depth for the object can be determined. Optionally, the backscattered photons can be focused via the optics to improve detection. Note that in implementations other than a single image, the source and detector may, for example, be moving with respect to the object, and/or there may be other or additional electronic, mechanical or electro-mechanical control to image the entire object.

The detector for the TOF system has an imaging pixel size of, for example, about 10 microns to about 100 microns, and a means for time resolution of a single photon detected in tens of picoseconds or possibly a few picoseconds. In addition, it would be preferred to include a photon correlator integrated with a read-out circuit. In one example, the photon correlator includes a time-to-voltage converter employing start/stop photon correlation.

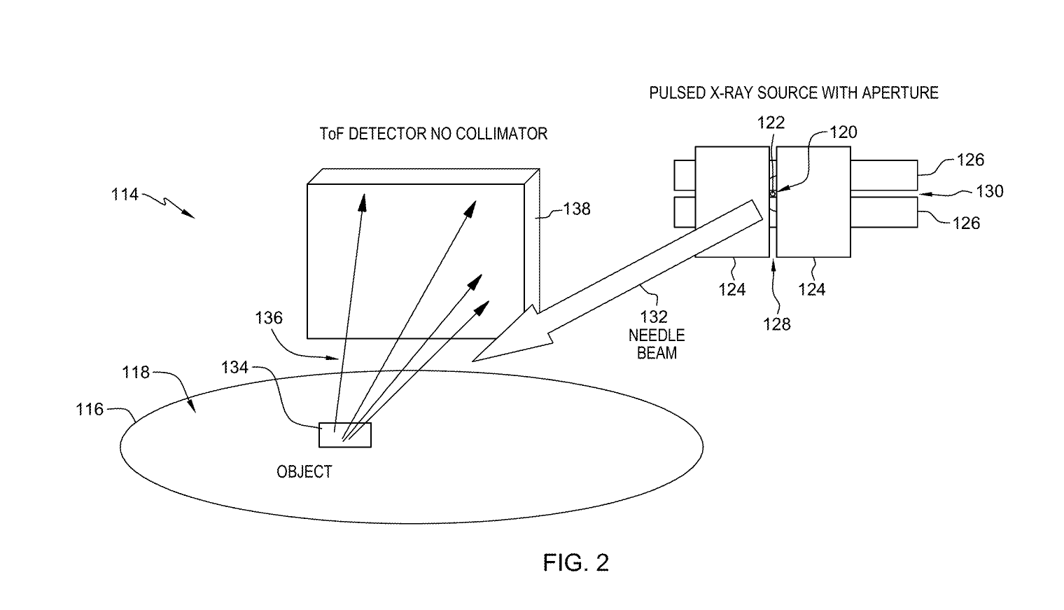

FIG. 2 depicts one simplified example of a system 114 for Time-of-Flight x-ray tomography, in accordance with one or more aspects of the present invention. The object 116 to be imaged is essentially flat and includes a circular top surface 118. The use of a simplified object is merely for ease of description. It will be understood that the system is designed to image three-dimensional objects. In this example, an aperture 120 is placed in front of a pulsed x-ray source 122, and formed, for example, with two pairs of members 124 and 126, each pair including a linear aperture (128 and 130, respectively) offset from the other by 90 degrees. The effect of the offset linear apertures is to create a “needle beam” 132 from the x-ray source. The needle beam impinges on a comparatively small portion 134 of the object's surface, the photons 136 of the needle beam backscattering and impinging randomly on a TOF detector 138. Note the absence of a collimator in front of the detector. The lack of any limiting apertures between the object and detector provides a high efficiency of detecting photons scattered from the object, resulting in a lower dose of x-rays for the object. Such a lower dose would be preferred for use with people, e.g., medical imaging. The potential downsides of this approach to achieve a lower dose includes the number of scanning exposures needed to get a complete image of the object, which takes time, and the complexity of a scan-control system, for example, a mechanical control system. In one example, where no collimator at the detector is present, if a N×M area of the object is imaged by P pulses, at least N×M×P x-ray pulses would be needed.

In the examples including a collimating aperture for the pulsed x-ray source, the size of the aperture is determined by desired resolution. For example, for a desired resolution of a=1 mm, the aperture size would be, for example, about 1 mm, and for the same in-depth resolution, the temporal resolution of the system would be, for example, about 2a/c=7 ps, where c is the speed of light. In addition, the aperture can be any shape desired.

In the case of FIG. 2, where no collimator at the detector is used and an aperture producing a “needle” beam is present, the dose is relatively low (e.g., about 10⁻⁴ rem=10⁻⁶ sv), the imaging time can be relatively slow when a mechanical scanner with moving parts is used to scan the beam against the object or object against the beam or combining both. Moving parts slow down the imaging process, and mechanical parts in general are prone to failure, but the system can be made expandable with multiple detectors at different locations to increase resolution and/or further reduce the dose. A possible application for this system is medical imaging, as dosage is key and taking more time is an acceptable tradeoff.

For comparison, the current annual maximum recommended dosage for an adult is 0.24 rem the dose from a film x-ray is about 10⁻³ to about 10⁻⁴ rem and a CT scan dose is about 0.1 to about 1 rem. Thus a single CT scan can easily exceed the recommended maximum dosage.

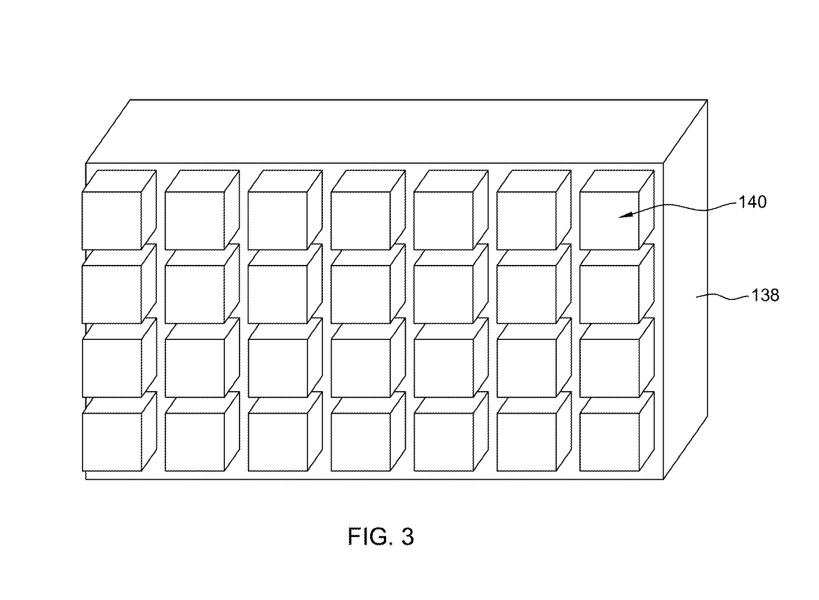

The detector shown in FIG. 3, includes an array of pixels 140 to obtain a two-dimensional image formed by the backscattered photons. In the case of FIG. 2, the entire image is used to form a single three-dimensional pixel, where the coordinate of the N×M array is defined by the position of the x-ray “needle” beam on the object, and the arrival times of the photons registered by the detector pixels define the depth coordinate D calculated from the setup geometry. The detector also includes some digital circuitry (not shown) for determining the TOF of each backscattered photon detected, for example, a start/stop implementation taking the form of a time-to-voltage converter with a programmable semiconductor circuit (e.g., field programmable gate array) or a dedicated specific functional circuit (application-specific IC—ASIC), which will generally be referred to herein as a “processor.” Where implemented as a semiconductor chip, the circuit can be silicon-based or III-V semiconductor material based (i.e., at least one element from each of groups III and V of the Periodic Table of Elements). Preferably, each pixel has a capacitance of less than about 20 fF to obtain fast temporal response and adequate signal-to-noise ratio. Each pixel is associated with its own detector, and the detectors operate independent of each other. Although integration with the detector of the circuit or other means used to determine the TOF is not required, integration is preferred in order to maximize temporal resolution of the pixels. In another example, the processor may take the form of a detector integrated with amplification-discrimination circuitry. In any case, the sequence of illuminating, detecting and determining a TOF is repeated until all portions of interest of the object have been imaged. In one example, a conventional CCD or CMOS pixel-based imager could be used for imaging larger objects (e.g., house, plane, boat, car) in conjunction with a scintillator, which includes a material that fluoresces when struck by a charged particle or high-energy photon. The scintillating material can be used to detect a backscattered photon. In response to detecting a photon, a means of determining the TOF for the detected photon is engaged (see options above).

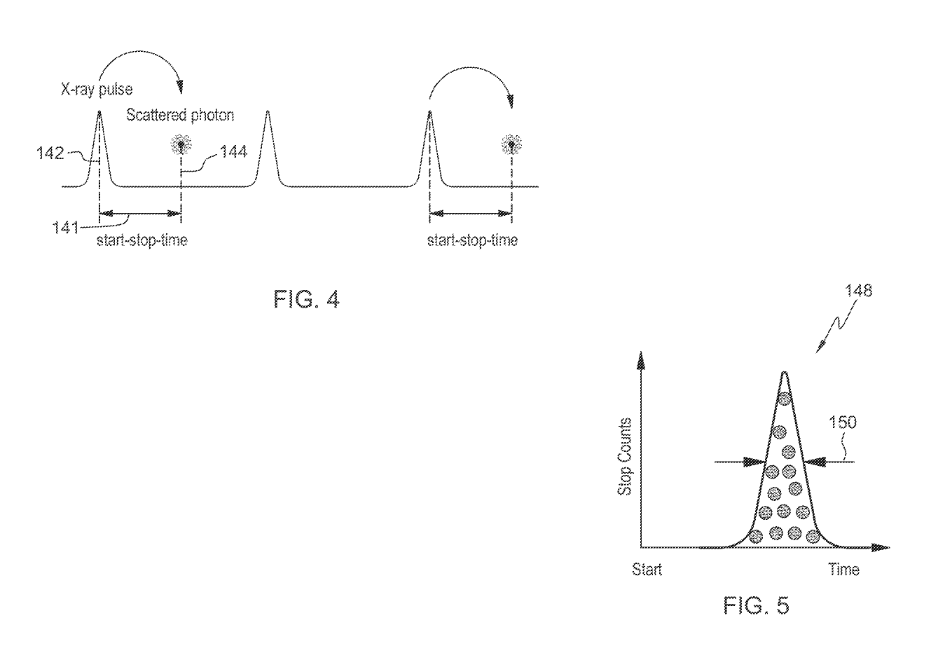

FIGS. 4 and 5 will now be referenced to describe the principles of start-stop single photon correlation. As shown in FIG. 4, an elapsed time 141 between a start time 142 (incident x-ray pulse) and a stop time 144 (detection) for the backscattered x-ray photons 146 is measured. As shown in FIG. 5, a graph 148 of distribution of start-stop times (141, FIG. 4) from a single scattering point indicates the number of backscattered photons arrived within the same start-stop time interval; the width of the distribution 150 corresponds to the time resolution of the system. A Time-of-Flight t=2a/c, where a is the depth of x-ray scattering in the object c=3×10⁻⁴ cm/s is the speed of light. A Time-of-Flight of one picosecond, for example, translates to a photon travelling length of 300 microns, which is halved to get the object depth, in this case 150 microns.

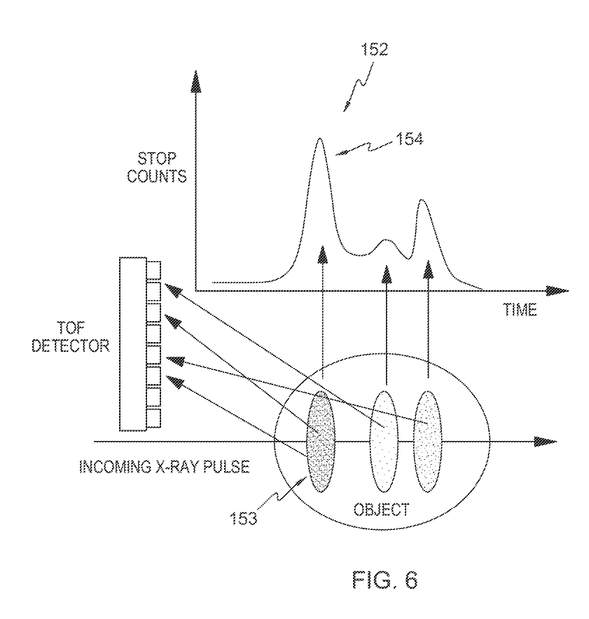

FIG. 6 depicts an example of a distribution of Time-of-Flights of x-ray photons 152 travelling from the source to the object and backscattered to the TOF detector. The number of backscattered photons is higher from the part of the object with higher density 153 and produces a higher peak 154 in the distribution 152. Alternatively, parts of the object with lower density, scatter less photons and produce lower peaks. In order to obtain high 3D image contrast, the pulsed X-ray source should provide photons that are strongly scattered by the object. The X-ray backscattering in materials and tissues is dominated by Compton scattering, which is most efficient in the photon energy range from about 20 keV to about 2 MeV.

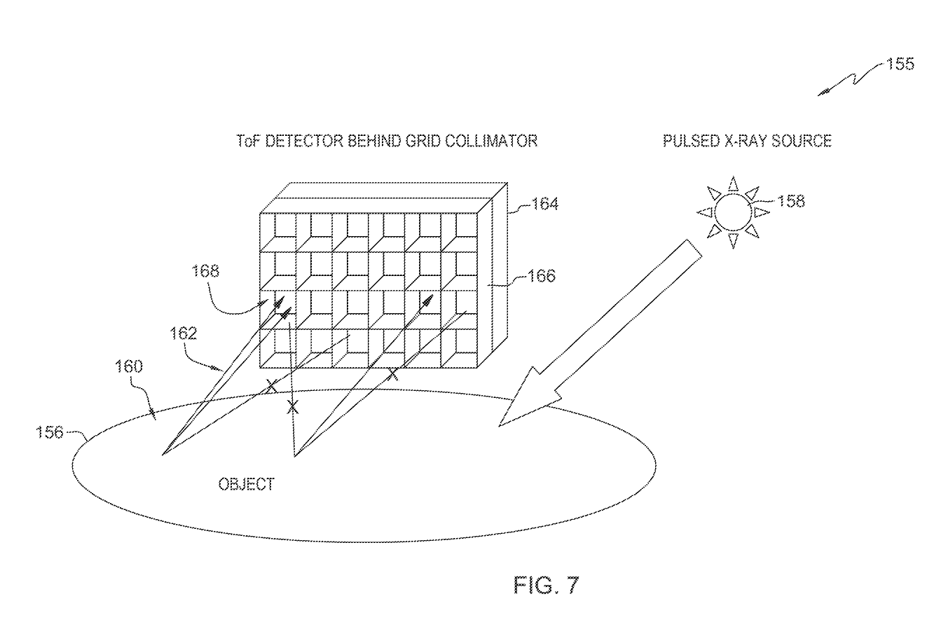

FIG. 7 depicts another simplified example of a system 155 for TOF x-ray tomography, in accordance with one or more aspects of the present invention. The object 156 to be imaged is the same as FIG. 2; that is, a simplified flat and circular object. In this example, there is no collimating aperture in front of the pulsed x-ray source 158, such that a maximum dose is applied to the top surface 160 of the object. Note, however, that illuminating the entire object surface leads to random backscattering of photons 162 with a lower probability of detection than the first example, such that a relatively higher irradiation dose is needed. Between a TOF detector 164, similar to that described with respect to FIG. 2, and the top surface of the object is a grid collimator 166. The grid collimator is a simplified example to convey the function needed, but optics capable of focusing the photons could instead be used, for example, capillary optics. As with the prior example, the photons of the x-ray pulse that impinge the object surface are backscattered toward the collimator. Only photons that impinge the collimator within one of the grid “boxes” (e.g., grid box 168) will reach the detector (i.e., photons scattered parallel to the collimator plates); all others will be absorbed by the collimator. With a high enough aspect ratio of the collimator, it is possible to achieve high resolution image of the object, for example, an aspect ratio of 100, translates to about 2 mm resolution when the detector is placed at 20 cm from the object. System 155 allows for obtaining a 3D image of the entire object with small number of x-ray pulses (though relatively high powered); no scanning or other mechanical motion is required.

In the examples including a collimator, the spacing of the plates and their length are generally determined by the desired resolution and the size of the detector. In one example, an object to be imaged is relatively small compared to the detector size, resulting in a resolution equal to the spacing between the plates, and each pixel behind the collimator “sees” a brick-shaped, rectangular volume of the object. In an opposite example, where the object is large in comparison to the detector, each cell of the detector “sees” a diverging volume of the object (similar in concept to the somewhat triangular image produced by ultrasound), such that resolution is determined by an angle (not distance) between the plates, and resolution is dependent on the distance to the detector. In a real-world system, the plate spacing would fall somewhere in between the two extremes given above.

In the case of FIG. 7, where no aperture is placed in front of the pulsed x-ray source and a grid collimator is placed in front of the detector, the effective x-ray dose is relatively high compared to the other examples (e.g., 100 rem), but the imaging time is relatively fast and there are no moving parts. The system of this example can be used as a high throughput system. In one example, this type of system can be used to image production line moving parts for defects (defectoscopy), since the x-ray dose is not critical for metals, but throughput is important.

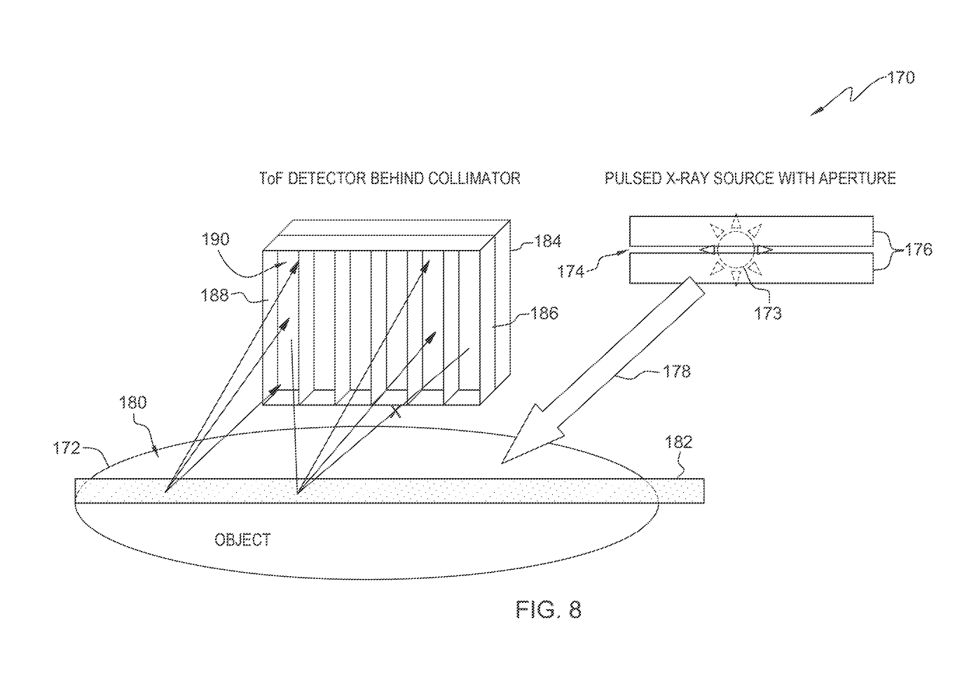

FIG. 8 depicts still another simplified example of a system 170 for TOF x-ray tomography, in accordance with one or more aspects of the present invention. As with the second example above, the object 172 to be imaged is the same as that of FIG. 2. In front of the pulsed x-ray source 173 is a horizontal aperture 174 created, for example, by a pair 176 of offset members, creating a horizontal slit through which the x-rays will pass, resulting in a planar x-ray beam 178 impinging on the surface 180 of the object. In other words, the system of this example obtains image slices (e.g., image slice 182) of the object. Between a TOF detector 184, as described previously, and the surface of the object is a linear collimator 186, the plates (e.g., plate 188) creating vertical detection areas (e.g., area 190). Note the slit in front of the pulsed x-ray source and the collimator plates are 90 degrees offset from each other. The slit could also be, for example, vertical with horizontal collimator plates. This third example is a compromise between dosage and detection, the first and second examples being more extreme.

In the case of FIG. 8, where a linear aperture and linear collimator are used, the dose and imaging speed are between that of the other two examples (e.g., a dose of about 0.1 rem). In other words, there is a trade-off to gain speed at the expense of a modest or reasonable increase in dosage. Since less than the entire object is imaged with each pulse, there would also need to be moving parts in the system (moving the object and/or moving around the object), for example, conventional mechanical or electromechanical systems. In one example, such a system may be used to image comparatively slow-moving parts and/or parts that are somewhat sensitive to radiation, for example, polymers.

In all three example systems above, for a N×M×D “three-dimensional image” with average Q-tone greyscale per point, a total of N×M×D×Q x-ray backscattered x-ray photons should be detected. A single element detector can detect one or less photons per cycle. In the first case of “needle beam” (FIG. 2), all the detector elements simultaneously detect photons scattered from one 3D pixel. In this case, use of the detector matrix provides the means to increase the solid angle for x-ray photon detection keeping the capacitance of the detector element low. The number of detector elements is irrelevant to the size of the 3D image, and defines the maximum number of photons that can be detected within one cycle. In the case of FIG. 7, with a grid collimator in front of the detector, the TOF detector array has possibly N×M elements or a multiple of N×M elements, and each detector element or a group of detector elements collect the signal from one 3D pixel. In the intermediate case of FIG. 8, the entire column as in the FIGURE (or, alternatively, row) of elements detects the scattered signal simultaneously from one 3D pixel, which is now defined by a planar x-ray beam and 2D array of collimators in front of the detector. Here, the number of detector elements in the column (or row) defines the maximum number of photons that can be detected within one cycle in 3D pixel. Likewise, the number of columns (or rows) in the matrix determines one of the dimensions of the image.

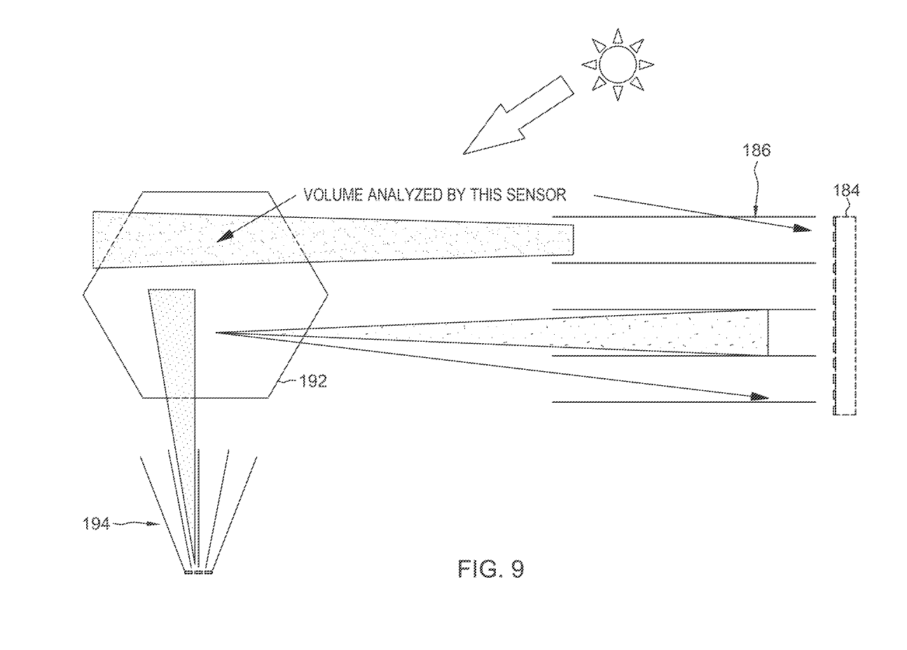

FIG. 9 depicts one example of a scattering point 192 of an object being imaged, with collimator 186 and TOF detector 184 of the example of FIG. 7, useful for an object comparable in size to the detector, and a fan-like collimator 194 useful for objects significantly larger than the size of the detector.

In a first aspect, disclosed above is a method of three-dimensional imaging. The method includes providing an x-ray source configured to produce pulsed x-rays, each pulse having a time duration of about 100 ps or faster, pulsing the x-ray source at least once to illuminate at least part of an object, detecting via a detector individual backscattered photon(s) from the object, and determining a length of time for an individual backscattered photon to travel from the x-ray source and backscattered to the photon detector.

In one example, the method may further include, for example, creating a needle beam from the pulsed x-ray source for illuminating the object. Creating the needle beam may include, for example, forming a collimating aperture in front of the x-ray source. In one example, forming the aperture may include, for example, providing a first pair of linear spaced members and a second pair of linear spaced members, the first and second pairs being offset from each other by about 90°. The image is formed by scanning the beam against the object and/or scanning the object against the beam. More generally, the beam is caused to illuminate the object.

In one example, the method of the first aspect may further include, for example, providing a grid collimator in front of the detector, the grid collimator including plates in a grid pattern, fully illuminating the object with a pulse or pulses of the x-ray source, with only photons backscattered parallel to and between the plates being detected.

In one example, the method of the first aspect may further include, for example, providing a linear collimator in front of the detector, the linear collimator including parallel plates. In addition, the pulsing of the method of the first aspect may include creating a planar beam to illuminate a planar band of the object, the planar beam being about 90° offset from the parallel plates, with only photons backscattered parallel to and between the parallel plates being detected. The image is formed by scanning the beam against the object, or scanning the object against the beam, or some combination of both. More generally, the beam is caused to illuminate the object.

In one example, the method of the first aspect may further include, for example, providing a fan-type collimator including plates angled outward from each other in a fan-type arrangement.

In a second aspect, disclosed above is a system for Time-of-Flight tomography. The system includes an x-ray source capable of producing pulsed x-rays with a pulse duration of about 100 ps or faster, a single-photon detector configured to detect individual photons backscattered from an object when present and illuminated by the x-ray source, the single-photon detector producing a two-dimensional image, and a processor for determining a Time-of-Flight of an individual backscattered photon from the x-ray source to the single-photon detector.

In one example, the x-ray source may include, for example, a synchrotron, a linear accelerator, a laser plasma source or a free-electron laser.

In one example, the processor in the system of the second aspect may include, for example, a time-to-voltage converter employing start-stop photon correlation.

In one example, the system of the second aspect may further include, for example, member(s) impervious to x-rays and arranged to create an aperture for the x-ray source, the aperture limiting an area of illumination of the object. In one example, the aperture forms a needle beam from pulsed x-rays. In another example, the aperture forms a planar x-ray beam from the pulsed x-rays.

In one example, where a planar x-ray beam is produced, the system of the second aspect may further include, for example, a linear collimator in front of the detector, the linear collimator including parallel plates impervious to x-rays and offset with regard to the aperture by about 90°, only photons scattered parallel to and between the parallel plates impinging on the single-photon detector.

In one example, the system of the second aspect may further include, for example, a grid collimator situated in front of the single-photon detector, the grid collimator including plates impervious to x-rays arranged in a grid pattern, with only photons scattered parallel to and between the plates reaching the single-photon detector.

In one example, the system of the second aspect may further include, for example, a fan-type collimator including plates angled outward from each other in a fan-type arrangement.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A method of three-dimensional imaging, the method comprising:
providing an x-ray source configured to produce pulsed x-rays, each pulse having a time duration of about 100 ps or faster;
pulsing the x-ray source at least once to illuminate at least part of an object;
detecting via a detector one or more individual backscattered photons from the object; and
determining a length of time for an individual photon to travel from the x-ray source and backscattered by the object to the photon detector.

2. The method of claim 1, further comprising:
creating a needle beam from the pulsed x-ray source for illuminating the object;
causing the needle beam to illuminate the object; and
scanning of the needle beam against the object and/or object against the beam.

3. The method of claim 2, wherein creating the needle beam comprises forming an aperture in front of the x-ray source.

4. The method of claim 3, wherein creating the aperture comprises providing a first pair of linear spaced members and a second pair of linear spaced members, the first and second pairs being offset from each other by about 90°.

5. The method of claim 1, further comprising providing a grid collimator in front of the detector, the grid collimator comprising a plurality of plates in a grid pattern, fully illuminating the object with a single pulse of the x-ray source, wherein only photons backscattered parallel to and between the plurality of plates are detected.

6. The method of claim 1, further comprising providing a linear collimator in front of the detector, the linear collimator comprising a plurality of parallel plates, wherein the pulsing comprises creating a planar beam to illuminate a planar band of the object, the planar beam being about 90° offset from the parallel plates, and wherein only photons scattered parallel to and between the plurality of parallel plates are detected.

7. The method of claim 6, further comprising causing the planar beam to illuminate the object.

8. The method of claim 1, further comprising providing a fan-type collimator comprising a plurality of plates angled outward in a fan-type arrangement.

9. A system for time-of-flight tomography, the system comprising;
an x-ray source capable of producing pulsed x-rays with a pulse duration of about 100 ps or faster;
a single-photon detector configured to detect individual photons backscattered from an object when present and illuminated by the x-ray source, the single-photon detector producing a two-dimensional image; and
a processor for determining a time-of-flight of an individual backscattered photon from the x-ray source to the single-photon detector.

10. The system of claim 9, wherein the x-ray source comprises one of a synchrotron, a linear accelerator, a laser plasma source and a free-electron laser.

11. The system of claim 9, wherein the processor comprises a time-to-voltage converter employing start-stop photon correlation.

12. The system of claim 9, further comprising one or more members impervious to x-rays and arranged to create an aperture for the x-ray source, the aperture limiting an area of illumination of the object.

13. The system of claim 12, wherein the aperture forms a needle beam from pulsed x-rays.

14. The system of claim 12, wherein the aperture forms a planar x-ray beam from the pulsed x-rays.

15. The system of claim 14, further comprising a linear collimator in front of the detector, the linear collimator comprising a plurality of parallel plates impervious to x-rays and offset with regard to the aperture by about 90°, wherein only photons scattered parallel to and between the plurality of parallel plates impinge on the single-photon detector.

16. The system of claim 9, further comprising a grid collimator situated in front of the single-photon detector, the grid collimator comprising a plurality of plates impervious to x-rays arranged in a grid pattern, wherein only photons scattered parallel to and between the plurality of plates reach the single-photon detector.

17. The system of claim 9, further comprising a fan-type collimator comprising a plurality of plates angled outward in a fan-type arrangement.
